(19) United States
(12) Patent Application Publication
(10) Pub. No.: US 2023/0037103 A1
(43) Pub. Date: Feb. 2, 2023

(54) USE OF A COMPOUND OF 4-(3-ETHOXY-4-HYDROXYPHENYL) ALKYL KETONE TYPE AGAINST BACTERIA OF THE BURKHOLDERIA CEPACIA COMPLEX

(71) Applicant: L’OREAL, Paris (FR)

(72) Inventors: Sylvie CUPFERMAN, Chevilly La Rue (FR); Florence MENARD-SZCZEBARA, Chevilly La Rue (FR)

(21) Appl. No.: 17/778,216

(22) PCT Filed: Nov. 26, 2020
(86) PCT No.: PCT/EP2020/083516

§ 371 Date: May 19, 2022

(30) Foreign Application Priority Data
Nov. 28, 2019 (FR) FR1913366

(51) Int. Cl. C07C 49/185; A01N 35/02; A01P 1/00; A61K 8/35; A61K 31/12; C02F 1/50

(52) U.S. Cl. CPC C07C 49/185; A01N 35/02; A01P 1/00; A61K 8/35; A61K 31/12; C02F 1/50; C02F 2303/04

(57) ABSTRACT

The present invention relates to the use of compounds of formula (I) in the treatment of products, in particular of aqueous or nonaqueous compositions, preferably water and aqueous compositions, against bacteria of the Burkholderia cepacia complex (Bcc), and also the use thereof in a process for treating an aqueous composition or water with respect to strains of the Bcc complex.

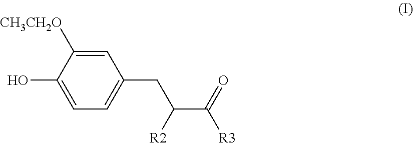

formula (I) wherein:
R2 represents a hydrogen atom or a methyl or ethyl radical;
R3 represents a linear C₁-C₁₂ alkyl radical (saturated), optionally substituted with a hydroxyl group; or a linear C₂-C₁₂ alkenyl radical (C═C unsaturated), optionally substituted with a hydroxyl group.

The present invention relates to the use of a compound of 4-(3-ethoxy-4-hydroxyphenyl) alkyl ketone type in the treatment of products or compositions which are aqueous or nonaqueous, preferably water and aqueous compositions, against bacteria of the Burkholderia cepacia complex (Bcc), and also the use thereof in a treatment process with respect to strains of the Bcc complex.

Antimicrobial agents are widely used in healthcare establishments, dwellings, farms and industries, for inhibiting the proliferation of damaging organisms and killing potential pathogens.

Microbial contamination during the production of an industrial product is common, even when starting ingredients placed in said product are “clean”, i.e. no contaminating microorganisms are present. Water, in particular, which is omnipresent in most cosmetic, pharmaceutical or food products, must be free of contaminating microorganisms. The cleanliness during production of these industrial products, the processing of the contents and the filling of the containers must be scrupulously monitored, as must the industrial equipment. Despite these precautions, the microbial integrity of the products generally requires the presence of one or more preservatives compatible with the product and the stability of the composition. The products must allow neither growth nor survival of contaminating microorganisms. Even if it is industrially possible to carry out a restrictive production in a sterile environment, maintaining the stability during use is problematic, since fingers, cosmetic applicators and even ambient air are not sterile. Preservatives are therefore required in order to reduce contamination with microorganisms by consumers during normal use. As a general rule, pathogenic microorganisms must be absent from all products sold, in particular cosmetic products (Kirk Othmer Encyclopedia, Cosmetics, Martin M. Rieger, 04/12/2000; https://doi.org/10.1002/0471238961.0315191318090507.a01 and standard ISO 17516 Cosmetics—Microbiology—Microbiological limits).

In industry, antimicrobial combinations are incorporated at low concentration in finished products and starting materials as preservatives for protecting against microbial contamination. The failure of these preservation systems can lead to a considerable economic loss and, depending on the contaminant, can present a risk to the health of the consumer. A wide range of yeasts, molds, fungi and bacteria may be responsible for product contaminations. Gram-negative bacteria can be encountered in industries, in particular pharmaceutical industries and cosmetic industries, with a predominance in product recalls concerning in particular bacteria of the Burkholderia cepacia complex (Jimenez, L., Microbial diversity in pharmaceutical product recalls and environments. PDA Journal of Pharmaceutical Science and Technology, 2007. 61(5): p. 383-399, Rushton, L. Rapid Alert System Weekly Notification Reports. 2005-2014).

Industry is seeking to formulate products comprising robust preservation systems with a broad antimicrobial spectrum, which prevent microbial growth and which do not result in the development of resistant microorganisms. This challenge becomes more complex because of restrictions imposed on the use of preservatives and the pressure exerted by consumers for a milder preservation of products, in particular cosmetic products. Over the past few years, safety problems linked to potential estrogenic activity and to skin sensitization problems have led to a significant reduction in the use of preservatives such as isothiazolinone, which are highly effective agents against bacteria of the Bcc complex (Rushton, L., et al., Key role for efflux in the preservative susceptibility and adaptive resistance of Burkholderia cepacia complex bacteria. Antimicrobial Agents and Chemotherapy, 2013. 57(7): p. 2972-2980). This underlines the need for new preservatives that are highly effective against the key risk species, such as the bacteria of the Bcc complex.

The Burkholderia cepacia complex (Bcc) comprises more than 20 closely related but genetically distinct species within the Burkholderia genus. As highly adaptable natural bacteria, Bccs have been studied for their potential biotechnological applications in plant promotion, bioremediation and biological control of plant pests (Mahenthiralingam, E., A. Baldwin, and C. G. Dowson, Burkholderia cepacia complex bacteria: Opportunistic pathogens with important natural biology. Journal of Applied Microbiology, 2008. 104(6): p. 1539-1551).

Bccs had been widely studied as opportunistic pathogens that can cause an infection in several hosts, such as chronic respiratory infections in individuals suffering from cystic fibrosis (CF) (Depoorter, E., et al., Burkholderia: an update on taxonomy and biotechnological potential as antibiotic producers. Applied Microbiology and Biotechnology, 2016. 100(12): p. 5215-5229). As industrial contaminants, Bcc bacteria have been isolated from petroleum products (White, J., et al., Culture-independent analysis of bacterial fuel contamination provides insight into the level of concordance with the standard industry practice of aerobic cultivation. Applied and Environmental Microbiology, 2011. 77(13): p. 4527-4538), (Álvarez-Lerma, F., et al., Moisturizing body milk as a reservoir of Burkholderia cepacia: Outbreak of nosocomial infection in a multidisciplinary intensive care unit. Critical Care, 2008. 12(1)), from pharmaceutical products and also cosmetic products, and also from toiletry products (Jimenez, L., Microbial diversity in pharmaceutical product recalls and environments. PDA Journal of Pharmaceutical Science and Technology, 2007. 61(5): p. 383-399). Bcc infection epidemics in vulnerable individuals, although rare, are the result of the use of contaminated industrial products [Álvarez-Lerma, F., et al., Moisturizing body milk as a reservoir of Burkholderia cepacia: Outbreak of nosocomial infection in a multidisciplinary intensive care unit. Critical Care, 2008. 12(1)., Kutty, P. K., et al., Multistate outbreak of Burkholderia cenocepacia colonization and infection associated with the use of intrinsically contaminated alcohol-free mouthwash. Chest, 2007. 132(6): p. 1825-1831], and these bacteria are now recognized as risk species in microbial contamination (Torbeck, L., et al., Burkholderia cepacia: This decision is overdue. PDA Journal of Pharmaceutical Science and Technology, 2011. 65(5): p. 535-543).

The ability of Bcc bacteria to survive as contaminants is partly due to a high innate antimicrobial resistance and to the ability to adapt to their environment (Mahenthiralingam, E., A. Baldwin, and C. G. Dowson, Burkholderia cepacia complex bacteria: Opportunistic pathogens with important natural biology. Journal of Applied Microbiology, 2008. 104(6): p. 1539-1551). A recent study of Bcc sensitivity to the main preservative groups used in industry has revealed differences in sensitivity between species, for example the levels of sodium benzoate and of benzethonium chloride used appear to be ineffective with respect to contamination by the bacteria of the Bcc complex [Rushton, L., et al., Key role for efflux in the preservative susceptibility and adaptive resistance of Burkholderia cepacia complex bacteria. Antimicrobial Agents and Chemotherapy, 2013. 57(7): p. 2972-2980]. In addition, isolated Bcc strains originating from industrial sources have shown an increased tolerance to a formaldehyde-releasing agent, suggesting Bcc emergence in industry, which is problematic (Rushton, L., et al., ibid). These intrinsically resistant opportunistic pathogens are responsible for or contribute to the contamination of industrial products.

Moreover, it is known that water is a vector for the propagation of pathogenic microorganisms; thus, contaminated waters can be responsible for numerous diseases.

Among the pathogenic microorganisms found in aqueous compositions and in water are the bacteria of the Bcc complex. These opportunistic bacteria therefore encompass a collection of aerobic Gram-negative pathogenic bacteria, most of which are opportunistic pathogens for human beings, animals and plants.

For public health reasons, there thus remains a real need to provide new active agents that are effective in the treatment of products that may be contaminated by bacteria of the Bcc complex, such as food, water and aqueous and anhydrous compositions. In particular, there remains a real need to find new active agents that make it possible i) either to prevent the contamination of industrial or domestic products, in particular water or aqueous or anhydrous compositions, by one or more microorganisms, including bacteria of the Burkholderia genus, ii) or to treat consumed products, in particular water or aqueous or anhydrous compositions, contaminated by one or more microorganisms, including bacteria of the Bcc complex. Said industrial or domestic products and particularly the water included in aqueous compositions for domestic or industrial uses, in particular products originating from industrial and domestic tanks, waters from aquatic media, swimming pool/spa waters, and air-conditioning system waters, and cosmetic, pharmaceutical and food products.

The object of the present invention is to satisfy these needs.

Surprisingly, the inventors have discovered that the use of at least one compound of formula (I) as defined below, in particular 4-(3-ethoxy-4-hydroxyphenyl) alkyl ketone or ethylzingerone (EZ), makes it possible to reduce the growth of bacteria of the Burkholderia cepacia complex of contaminated products. The use of at least one compound of formula (I) as defined below, in particular 4-(3-ethoxy-4-hydroxyphenyl) alkyl ketone or ethylzingerone (EZ), makes it possible to prevent the contamination, by bacteria of the Burkholderia cepacia complex, of products that may be in contact with one or more microorganisms, including bacteria of the Burkholderia genus, of the Bcc complex. Indeed, it appears that the compounds of formula (I) act as a preservative with a high activity against bacteria of the Burkholderia cepacia complex leading in particular to a reduction in viability of several log after several hours (6 log in 4 hours).

The compounds of formula (I), in particular EZ, are new preservatives which unexpectedly exert an acute activity against Burkholderia, based on a new multifactorial action mode and, furthermore, do not promote resistance to these resilient Gram-negative bacteria, on products that may be contaminated by bacteria of the Bcc complex, such as water, industrial or domestic aqueous compositions, industrial or domestic equipment, such as industrial or domestic preparation tanks, the solvents used in industrial or domestic preparations or in industrial equipment, or even industrial or domestic anhydrous compositions.

In addition to the treatment of products that may be contaminated by bacteria of the Bcc complex, the use of compounds of formula (I) also makes it possible to treat the biofilm present at the surface of devices in contact with water, such as for example water pipes, or at the surface of industrial or domestic tanks.

A subject of the invention is also a process for treating, in particular for preserving, a product that may be contaminated by bacteria of the Burkholderia cepacia complex, in particular a composition, comprising particularly a physiologically acceptable medium, more particularly a cosmetic or pharmaceutical composition, characterized in that it consists in incorporating into said product, during the production thereof, or after the production thereof and preferably before storage, one or more compounds of formula (I), preferably EZ.

Thus, the present invention relates to the use of at least one compound of formula (I) and also the organic or mineral acid or base salts thereof, or solvates thereof such as hydrates:

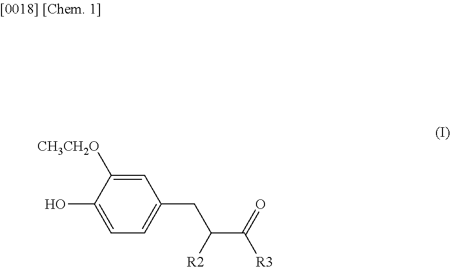

wherein:
R2 represents a hydrogen atom or a methyl or ethyl radical;
R3 represents a linear C₁-C₁₂ alkyl radical (saturated), optionally substituted with a hydroxyl group; or a linear C₂-C₁₂ alkenyl radical (C═C unsaturated), optionally substituted with a hydroxyl group;
   against bacteria of the Burkholderia cepacia complex, in particular in the treatment of products that may be contaminated by bacteria of the Bcc complex, such as water, aqueous compositions, anhydrous compositions, equipment, that may be industrial or domestic, in particular for food, cosmetic or pharmaceutical purposes, the solvents used in industrial or domestic preparations, preferably aqueous compositions or water, against bacteria of the Burkholderia cepacia complex.

The present invention also relates to a process for treating products that may be contaminated by bacteria of the Bcc complex, in particular for treating i) water or an aqueous composition, ii) equipment, that may be industrial or domestic, such as industrial or domestic food, cosmetic or pharmaceutical preparation tanks, iii) the solvents used in industrial or domestic preparations, and iv) industrial or domestic anhydrous compositions, comprising at least one step of bringing into contact one or more compounds of formula (I) as defined above, in particular bringing into contact with i) water or aqueous compositions, ii) equipment, that may be industrial or domestic, preferably for cosmetic, pharmaceutical or food purposes, iii) solvents used in industrial or domestic preparations, and iv) industrial or domestic anhydrous compositions.

More particularly, the present invention relates to a process for treating water or an aqueous composition, comprising at least one step of bringing a sample of water or of an aqueous composition to be treated or of a stream of water to be treated, that is continuous or batchwise (batch-type), said water to be treated being chosen from domestic or industrial waters, waters from aquatic media, swimming pool/spa waters, and waters from air-conditioning systems, into contact with one or more compounds of formula (I) as defined above.

The present invention also relates to the treatment of waters contained in food, cosmetic or pharmaceutical products, preferably cosmetic products, against bacteria of the Burkholderia cepacia complex.

The present invention also relates to the treatment of “anhydrous” products or compositions, i.e. having an amount of water of less than 5%, preferably less than or equal to 3%, even more particularly less than or equal to 1%, in particular free of water, against bacteria of the Burkholderia cepacia complex. These anhydrous products may be food, cosmetic or pharmaceutical products, preferably cosmetic products.

The present invention also relates to the treatment of aqueous or anhydrous non-food, cosmetic or pharmaceutical products against bacteria of the Burkholderia cepacia complex.

### Definitions

The expression “treatment of a product or treatment of products” is intended to mean a treatment, of the product(s), against bacteria of the Burkholderia cepacia complex, consisting in adding a substance to a sample of product(s) to be treated, continuously or batchwise (batch-type), for the purpose either of preventing the contamination of said product(s) by bacteria of the Burkholderia cepacia complex or of partially or totally decontaminating said product(s) to be treated, with respect to bacteria of the Burkholderia cepacia complex.

The term “water treatment” is intended to mean a continuous or batchwise (batch-type) treatment against bacteria of the Burkholderia cepacia complex, which consists in adding a substance to a sample of water to be treated or to a stream of water to be treated for the purpose either of preventing the contamination of the water by bacteria of the Burkholderia cepacia complex or of partially or totally decontaminating said water to be treated, with respect to bacteria of the Burkholderia cepacia complex.

Preferably, the treatment of water and of the aqueous compositions performed in the context of the present invention consists in adding, continuously or batchwise, a substance (a compound of formula (I) as defined above and in particular EZ) to a sample of water to be treated or to a stream of water to be treated in order to partially or totally decontaminate said water and aqueous compositions to be treated, with respect to a contaminating agent.

The contaminating agent according to the invention comprises bacteria of the Burkholderia cepacia complex.

The term “waters from aquatic media” is intended to mean the waters of lakes, tributary rivers, pools, mainstem rivers, sea or ocean bathing areas, underground waters such as well waters and groundwaters, and aquarium waters.

The term “aqueous composition” is intended to mean a composition which comprises at least 5% of water, in particular between 5% and 99.9%, more particularly between 10% and 90% of water, preferentially between 20% 80% of water, more preferentially between 30% and 70% of water, and optionally comprises one or more organic solvents, in particular those defined below, i.e. organic solvents having Hansen solubility space parameters such that: 14.5<δa<30 and 15<δd<22. The aqueous composition is more particularly aqueous-alcoholic, i.e. it comprises, in addition to the water, at least one (C₂-C₆)alkanol such as ethanol or a polyhydroxy(C₂-C₆)alkanol such as glycerol.

Said “aqueous” composition can also comprise one or more fatty substances, in particular liquid at ambient temperature and atmospheric pressure, such as oils, in particular natural oils. The aqueous composition can comprise other ingredients conventionally used in foods, cosmetics or pharmaceuticals, such as those chosen from anionic, nonionic, cationic or zwitterionic surfactants, fatty substances such as oils, which are natural or synthetic, or derived from hydrocarbons, natural or non-natural, anionic, nonionic, cationic or zwitterionic, associative or non-associative thickening or non-thickening polymers, dyes and pigments.

The term “anhydrous” composition is intended to mean a composition which comprises an amount of water of less than 5%, preferably less than or equal to 3%, even more particularly less than or equal to 1%, in particular which is free of water.

The anhydrous composition can comprise other ingredients conventionally used in foods, cosmetics or pharmaceuticals, such as those chosen from i) anionic, nonionic, cationic or zwitterionic surfactants, ii) fatty substances, in particular oils, which are natural or synthetic, or derived from hydrocarbons, iii) natural or non-natural, anionic, nonionic, cationic or zwitterionic, associative or non-associative thickening or non-thickening polymers, iv) dyes, v) pigments, vi) fillers and vii) their mixture.

Said anhydrous composition can also comprise one or more fatty substances, in particular liquid at ambient temperature and atmospheric pressure, such as oils, in particular natural oils.

For the purposes of the present invention, the “domestic or industrial waters” comprise waste waters before they have been treated in a purification plant, waters undergoing treatment in a purification plant, waters before they have been treated in a drinking water plant, waters undergoing treatment in a drinking water plant, and also waters circulating in potable or non-potable urban networks, for instance waters circulating in pipes.

The term “biofilm” is intended to mean a deposit of microorganisms at the surface of a device in contact with water or solvents, this deposit being formed in particular by adsorption of microorganisms at the surface of said device. As device which comes into contact with the water or solvents, mention may for example be made of: pipes, in particular water pipes, cooling towers, etc.

The term “industrial or domestic equipment” is intended to mean any industrial or domestic tools or machines which help to create or to prepare, treat, convert or store natural or synthetic chemical products, biochemical products or biological products. Said equipment may be adjusted to the pressure, i.e. to a pressure below or above atmospheric pressure, preferably to a pressure greater than 1 atm, and to a high or low temperature, i.e. to a temperature above or below ambient temperature 25° C., preferably to a temperature above 30° C. In particular, said equipment is partly constituted of metal, glass, ceramic, and/or plastic such as PVC, or rubber. Preferably, the equipment of the invention is industrial. Preferentially, the equipment of the invention is constituted in part (which may be tanks or reactors, pipework, etc.) of metal such as aluminum, iron, copper, zinc, steel, stainless steel, cast iron, more preferentially of steel or stainless steel.

The term “tank” is intended to mean any domestic or industrial container for the preparation, treatment, conversion or storage of natural or synthetic chemical products, biochemical products or biological products. Said container may be adjusted to the pressure, i.e. to a pressure above or below atmospheric pressure, preferably to a pressure greater than 1 atm, and to a high or low temperature, i.e. to a temperature above or below ambient temperature 25° C., preferably to a temperature above 30° C. In particular, said container is constituted of metal, glass, ceramic, and/or plastic such as; the tank is preferably made of metal. Preferably, the tank in the invention is an industrial tank. Preferentially, the tank of the invention is made of metal such as aluminum, iron, copper, zinc, steel, stainless steel, cast iron, more preferentially of steel or stainless steel.

### DETAILED DESCRIPTION OF THE INVENTION

Use

The compound of the following formula (I) and also the organic or mineral acid or base salts thereof, or solvates thereof such as hydrates:

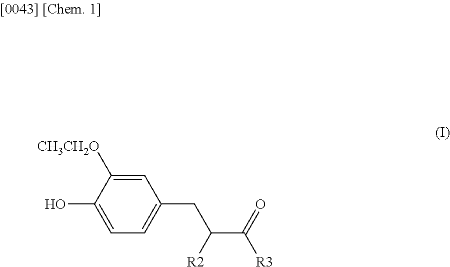

formula (I) wherein:
R2 represents a hydrogen atom or a methyl or ethyl radical;
R3 represents a linear C₁-C₁₂ alkyl radical (saturated), optionally substituted with a hydroxyl group; or a linear C₂-C₁₂ alkenyl radical (C═C unsaturated), optionally substituted with a hydroxyl group.

Preferably, the compounds correspond to formula (I), wherein:
R2 is chosen from a hydrogen atom and a CH₃ group; even better still, R2 represents a hydrogen atom; and/or
R3 represents (i) a C₁-C₁₀alkyl radical; (ii) a C₂-C₁₀ alkenyl radical, in particular a —CH═CH—R4 radical with R4 representing a linear C₁-C₆ alkyl radical; or else (iii) a hydroxyalkyl radical having the structure —CH₂—CH(OH)—R5 with R5 representing a linear C₁-C₁₀, preferably C₄-C₁₀, alkyl radical.

A mixture of compounds of formula (I) can be used.

Mention may particularly be made of the following compounds of formula (I):

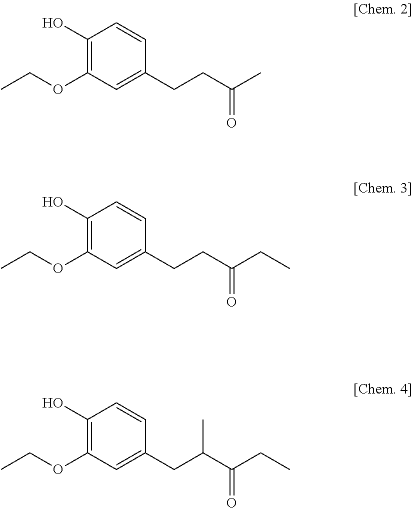

The compounds of formula (I) can be readily prepared by those skilled in the art on the basis of their general knowledge. Mention may be made especially of the following bibliographic references: J. Asian Natural Products Research, 2006, 8(8), 683-688; Helv. Chimica Acta, 2006, 89(3), 483-495; Chem. Pharm. Bull., 2006, 54(3), 377-379; and Bioorg. Med. Chem. Lett., 2004, 14(5), 1287-1289.

They may thus be prepared from ethylvanillin, in the manner described in the published patent application WO 2011/039445.

In one preferred embodiment of the present invention, use is made of 4-(3-ethoxy-4-hydroxyphenyl)butan-2-one having the following formula:

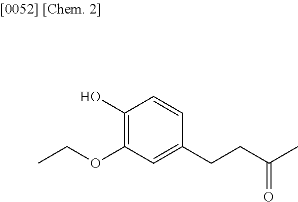

The compounds of formula (I) can be used in combination with at least one active agent chosen from anionic, cationic, nonionic or zwitterionic surfactants, descaling agents, coagulants, and mixtures thereof.

In one preferred embodiment, the compounds of formula (I) are used in combination with an effective amount of at least one organic solvent having solubility parameters according to the Hansen solubility space such that: 14.5<δ a<30 and 15<δ d<22.

Advantageously, the organic solvents used in the context of the present invention can be chosen from those described in the published patent application WO 2012/130953.

The total solubility parameter 6 according to the Hansen solubility space is defined in the article “Solubility parameter values” by Eric A. Grulke in the book “Polymer Handbook”, 3rd Edition, Chapter VII, pages 519-559, by the relationship:

δ=(δ²=δ_{d}²+δₚ²+δₕ²)^{1/2} [Math. 1]

wherein
δ d characterizes the London dispersion forces derived from the formation of dipoles induced during molecular impacts,
δ p characterizes the Debye interaction forces between permanent dipoles,
δ h characterizes the specific interaction forces (such as hydrogen bonding, acid/base, donor/acceptor, etc.). The definition of solvents in the Hansen three-dimensional solubility space is described in the article by C. M. Hansen: “The three dimensional solubility parameters” J. Paint Technol. 39, 105 (1967).

The parameter δ a is defined by the following relationship:

δₐ²=δₚ²+δₕ²=δ²−δ_{d}² [Math. 2]

The parameters δ d, δ p, δ h s and δ a are expressed in (J/cm3)½.

Preferably, the organic solvent has solubility parameters such that 14.5<δ a<28 and 15<δ d<20.

The organic solvent used according to the invention can be chosen from ethanol (δ a=20.20; δ d=15.10), 1,2-propylene glycol (δ a=25.00; δ d=16.00), 1,3-propanediol (δ a=26.32; δ d=18.00), PEG-8 (polyethylene glycol containing 8 ethylene glycol units) (δ a=14.80; δ d=17.90), propylene carbonate (δ a=18.46; δ d=20.00), dipropylene glycol (δ a=19.48; δ d=16.20), 1,2-hexylene glycol (δ a=19.20; δ d=16.40), PEG-4 (δ a=18.60; δ d=18.00).

Preferably, the organic solvent is chosen from ethanol, 1,2-propylene glycol, 1,3-propanediol, PEG-8 and propylene carbonate.

The solvent may be used in a content ranging from 0.05% to 10% by weight relative to the total weight of the water to be treated comprising bacteria of the Burkholderia cepacia complex Bcc, preferably ranging from 0.1% to 5% by weight and preferentially ranging from 0.1% to 2.5% by weight relative to the total weight of the water to be treated comprising bacteria of the Burkholderia cepacia complex Bcc.

The compounds of formula (I), alone or as a mixture, can be used in a proportion of at least 0.06% by weight, preferably at least 0.1% by weight, even better still at least 0.5% by weight relative to the total weight of water to be treated comprising bacteria of the Burkholderia cepacia complex Bcc.

In one preferred embodiment, the compounds of formula (I), alone or as a mixture, can be used in a proportion of at least 1% by weight relative to the total weight of water to be treated comprising bacteria of the Burkholderia cepacia complex. The compounds of formula (I), alone or as a mixture, can be used in a concentration ranging from 0.06% to 10% by weight, preferably from 0.06% to 10% by weight, even better still from 0.06% to 5% by weight relative to the total weight of water to be treated comprising bacteria of the Burkholderia cepacia complex. In one preferred embodiment, the compounds of formula (I), alone or as a mixture, can be used in a concentration ranging from 0.05% to 2%, preferably ranging from 0.1% to 1% by weight relative to the total weight of water to be treated comprising bacteria of the Burkholderia cepacia complex.

Process for Treating Water Against the of the Burkholderia cepacia Complex

Preferably, said step of bringing the water to be treated and the aqueous composition into contact with the compound of formula (I) can in particular be carried out by injection in liquid form of said compound, by passage through a filter or a filtering cartridge comprising said compound, or by administration in solid form of said compound notably in the form of granules, lozenges or pellets.

Process for Treating a Product that May be Contaminated by Bacteria of the Burkholderia cepacia Complex

According to one particular embodiment of the invention, the process for treating—in particular for preserving—a product that may be soiled by bacteria of the Burkholderia cepacia complex comprises the step of incorporating one or more compounds of formula (I) as defined above, preferably EZ, during the phase of producing said product.

The incorporation can be carried out by introducing into the preparation tank, beforehand, one or more compounds of formula (I), preferably EZ, preferably at a temperature of less than or equal to 80° C., better still at ambient temperature (25° C.), and particularly at atmospheric pressure, then introducing into said tank the product to be preserved, or the ingredients required to produce said product. According to one variant, each ingredient required for the production of the product was treated beforehand with one or more compounds of formula (I), in particular EZ, or else each ingredient is introduced as a mixture with one or more compounds of formula (I), in particular EZ. The product is then preserved and made available to the user in the presence of one or more compounds of formula (I), in particular EZ.

According to another particular embodiment of the invention, the process for preserving a product that may be soiled by bacteria of the Burkholderia cepacia complex comprises the step of incorporating one or more compounds of formula (I) as defined above, preferably EZ, after the phase of producing said product. The product, once produced, is brought into contact with one or more compounds of formula (I), in particular EZ, preferably at a temperature of less than or equal to 80° C., better still at ambient temperature (25° C.), and particularly at atmospheric pressure, then the product is preserved and made available to the user in the presence of one or more compounds of formula (I), in particular EZ.

According to yet another particular embodiment of the invention, the process for preserving a product that may be soiled by bacteria of the Burkholderia cepacia complex comprises the step of incorporating one or more compounds of formula (I) as defined above, preferably EZ, after the phase of producing said product. The product, once produced, is stored under an inert atmosphere, then once opened by the user it is brought into contact with one or more compounds of formula (I), in particular EZ, preferably at ambient (25° C.), and at atmospheric pressure, then the product is preserved in the presence of one or more compounds of formula (I), in particular EZ.

Preferably, the product is a composition, particularly comprising a physiologically acceptable medium, more particularly a cosmetic or pharmaceutical composition.

According to another embodiment of the invention, the product of the preserving process is not cosmetic.

The expressions “between . . . and . . . ” and “ranging from . . . to . . . ”, “at least . . . ” or “at most” should be understood as being limits inclusive, unless otherwise specified.

The examples and figures that follow are provided as illustrations which do not limit the field of the invention.

### EXAMPLES

Sensitivity Test

The minimum inhibitory concentration (MIC) and the minimum bactericidal concentration (MBC) of compounds of formula (I) of the invention, in particular EZ, were determined by standardized agar dilution and broth dilution tests, described by Rushton et al. Key role for efflux in the preservative susceptibility and adaptive resistance of Burkholderia cepacia complex bacteria. Antimicrobial Agents and Chemotherapy, 2013. 57(7): p. 2972-2980 using TSA/TSB. The culture was carried out at 30° C. in order to be representative of the production and storage of industrial products. Preserving stock solutions (50% w/v) were prepared in DMSO (Sigma-Aldrich, United Kingdom) and volumes were added to the medium so as to achieve the test concentration. The final concentration of DMSO in the presence of the bacterium was verified to be non-toxic (not exceeding 4% v/v) and was included as control condition in the tests in order to eliminate the effect on growth. The MIC is defined as the lowest concentration of preservative at which there is an 80% reduction in the liquid OD in culture (at 630 nm), or no visible growth of the organisms tested on an agar medium. The MBC was determined as being the lowest concentration for causing a 99% destruction rate, and for which the growth on the recovery medium (TSA) stopped. The preservatives were inactivated before the recovery and counting of the surviving organisms tested, by dilution in a neutralizing solution containing 1.5% v/v of Tween 80 containing 3% of lecithin, as described in the scientific publication by Rushton et al. (Rushton et al, ibid). The efficacy and toxicity of the neutralizing solution were evaluated before the experiment, as described in the scientific publication by Lear, J. C., et al. (J. C., et al., Chloroxylenol- and triclosan-tolerant bacteria from industrial sources—Susceptibility to antibiotics and other biocides. International Biodeterioration and Biodegradation, 2006. 57(1): p. 51-56).

### Example 1—Evaluation of the Inhibitory Activity of a Compound of Formula (I) with Respect to the Burkholderia cepacia Complex

Bacterial Strains and Culture Conditions.

A panel of 59 Burkholderia used to profile the sensitivity to the compounds of formula (I), in particular EZ, was established. The collection comprised 20 of the current groups of species of the Burkholderia cepacia complex (Bcc), reference strains for the panel of Bcc experimental strains (Mahenthiralingam, E., et al., Diagnostically and experimentally useful panel of strains from the Burkholderia cepacia complex. Journal of Clinical Microbiology, 2000. 38(2): p. 910-913) and 39 strains previously profiled for sensitivity to the preservative by Rushton et al. (Rushton, L., et al., Key role for efflux in the preservative susceptibility and adaptive resistance of Burkholderia cepacia complex bacteria. Antimicrobial Agents and Chemotherapy, 2013. 57(7): p. 2972-2980). The strains of the Burkholderia panel were isolated from strains of clinical (n=28), environmental (n=23) and industrial (n=8) origin. The strains were cultured once at 30° C. on tryptone soy (TSB/TSA) (Oxoid Ltd, United Kingdom). The strains were stored in TSB containing 8% v/v of dimethyl sulfoxide (DMSO) (Sigma-Aldrich, UK) at −80° C.

TABLE 1
S1. Burkholderia strains tested

| | | |
|---|---|---|
| | BCCM/LMG culture collection | |
| Name of species | accession number | Isolation source |
| and strain | (Other strain designations) | code |

B. ambifaria

| | | |
|---|---|---|
| AMMD ^{ESP a} | LMG 19182 ^{T} | ENV |
| BCC0267 ^{ESP a} | LMG 19467 | CLIN |
| BCC0338 ^{a} | LMG 17828 | ENV |

B. anthina

| | | |
|---|---|---|
| BCC0635 ^{ESP a} | LMG 16670 | ENV |
| BCC0639 ^{ESP a} | LMG 20980 ^{T} | ENV |
| BCC0485 ^{ESP} | LMG 21821 | CLIN |

B. arboris

| | | |
|---|---|---|
| BCC0049 ^{a} | — | CLIN |
| BCC1306 ^{a} | — | ENVI |
| BCC1310 ^{a} | — | ENVI |

B. cenocepacia III-A

| | | |
|---|---|---|
| BCC0018 ^{ESP a} | LMG 16659 | CLIN |
| J2315 ^{ESP} | LMG 16856 ^{T} | CLIN |

B. cenocepacia III-B

| | | |
|---|---|---|
| HI2424 ^{a} | — | ENV |

B. cepacia

| | | |
|---|---|---|
| BCC0001 ^{ESP a} | LMG 1222 ^{T} | ENV |
| BCC0002 ^{ESP a} | LMG 2161 | ENV |
| BCC0003 ^{ESP a} | LMG 18821 | CLIN |

B. contaminans

| | | |
|---|---|---|
| SAR-1 ^{a} | LMG 23255 | CLIN |
| BCC1315 ^{a} | — | ENVI |
| BCC0339 | — | CLIN |

B. diffusa

| | | |
|---|---|---|
| BCC0106 ^{a} | LMG 24286 | CLIN |
| BCC0169 ^{a} | (ATCC 29352) | ENV |
| AU1075 ^{a} | LMG 24085 | CLIN |

B. dolosa

| | | |
|---|---|---|
| BCC0161 | FC0380 | CLIN |
| AU0645 ^{ESP a} | LMG 18943 ^{T} | CLIN |
| AU3556 ^{a} | — | CLIN |

B. lata

| | | |
|---|---|---|
| Strain 383 ^{a} | LMG 22485 ^{T} | EW |
| BCC1296 ^{a} | — | ENVI |
| BCC1406 ^{a} | — | ENVI |

B. latens

| | | |
|---|---|---|
| BCC1625 | LMG 24064 ^{T} | CLIN |
| BCC1626 | LMG 24264 | CLIN |
| BCC1892 | LMG 24265 | CLIN |

B. metallica

| | | |
|---|---|---|
| BCC0095 ^{a} | — | CLIN |
| AU0553 ^{a} | LMG 24068 ^{T} | CLIN |

B. multivorans

| | | |
|---|---|---|
| BCC0149 | | CLIN |
| ATCC17616 ^{ESP a} | LMG 17588 | ENV |
| BCC1560 ^{a} | — | ENVI |

B. pseudomultivorans

| | | |
|---|---|---|
| BCC1884 | LMG 26883 | CLIN |
| BCC1191 ^{a} | D1443 | CLIN |

B. pyrrocinia

| | | |
|---|---|---|
| BCC0171 ^{ESP a} | LMG 21822 | ENV |
| BCC0180 ^{T ESP a} | LMG 14191 ^{T} | ENV |
| BCC0476 ^{ESP a} | LMG 21823 | ENV |

B. seminalis

| | | |
|---|---|---|
| BCC1627 ^{T} | LMG 24067 | CLIN |
| BCC1628 | LMG 19587 | ENV |
| BCC1893 | LMG 24272 | CLIN |

B. stabilis

| | | |
|---|---|---|
| BCC0023^{ESF a} | LMG 14294 ^{T} | CLN |
| BCC0286 ^{a} | (ATCC 35254) | ENV |
| AU6735^{a} | — | CLIN |

B. stagnalis

| | | |
|---|---|---|
| BCC1350 | HI3541 | ENV |
| BCC1887 ^{T} | LMG 28156 | ENV |
| BCC1896 | LMG 28157 | ENV |

B. territorii

| | | |
|---|---|---|
| BC1888 ^{T} | LMG 28158 | ENV |
| BCC1897 | LMG 28159 | ENV |

B. ubonensis

| | | |
|---|---|---|
| BCC1603 ^{a} | LMG 20358 | ENV |

B. vietnamiensis

| | | |
|---|---|---|
| BCC0028 ^{ESP A} | LMG 16232 | CLIN |
| BCC0195 | LMG 22486, G4 | ENV |
| BCC1309 ^{a} | — | ENVI |

B. gladioli

| | | |
|---|---|---|
| BCC1317 ^{a} | | ENVI |
| BCC0238 | MA4 | CLIN |
| BCC0507 | PF(#428) | CLIN |

B. plantarii

| | | |
|---|---|---|
| BCC0777 | LMG 9035 | ENV |

BCCM/LMG, Belgian co-ordinated collections of micro-organisms, Ghent.
ATCC, American type culture collection BCC.
CLIN, clinical;
ENV, environmental;
ENVI, environmental industrial.
T Type strain.
ESP experimental panel of experimental Burkholderia cepacia complex strains

The compounds of formula (I), in particular EZ, are very efficacious against bacteria of the Burkholderia cepacia complex Bcc. The compounds of the invention, in particular EZ, demonstrated excellent efficacy against a diversified panel of 57 Burkholderia strains representing species commonly encountered as contaminants.

In FIG. 1,
the center lines indicate the medians; the limits of the “box” indicate the 25th and 75th % determined by the free statistical software R; the Whiskers (i.e. method of representation of data in the box plot format—rectangle ranging from the first quartile to the third quartile cut by the median in purple on the graph above) extend over 1.5 times the interquartile range of the 25th and 75th %;
the data points are represented in the form of open circles;
the aberrant values are represented by dots;
the crosses represent examples of means; and
the width of the boxes is proportional to the square root of the size of the sample.

It appears that the compounds of formula (I) of the invention and in particular EZ act as a preservative with a high activity against bacteria of the Burkholderia cepacia complex leading in particular to a reduction in viability of several log after several hours (6 log in 4 hours).

All these experimental results show that the compounds of formula (I), in particular EZ, have a very significant inhibitory activity with respect to bacteria of the Burkholderia cepacia complex.

1. A process for treating a product that may be contaminated by bacteria of the Burkholderia cepacia complex (Bcc) which comprises contacting the product with an amount effective for protecting the product against bacteria of the Burkholderia cepacia complex of at least one compound of formula (I), an organic or mineral acid or base salt thereof, a solvate thereof or a mixture thereof: 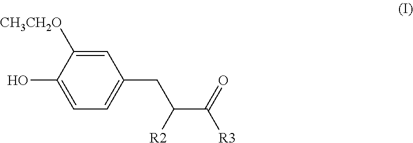
formula (I) wherein:
R2 represents a hydrogen atom or a methyl or ethyl radical;
R3 represents a linear C₁-C₁₂ alkyl radical (saturated), optionally substituted with a hydroxyl group; or a linear C₂-C₁₂ alkenyl radical (C═C unsaturated), optionally substituted with a hydroxyl group,
thereby protecting the product against bacteria of the Burkholderia cepacia complex (Bcc).

2. The process as claimed in claim 1, for preventing the contamination of a product that may be contaminated by bacteria of the Bcc complex selected from the group of i) of water or an aqueous composition, ii) of equipment for food, cosmetic or pharmaceutical purposes, iii) of solvents used in industrial or domestic preparations, and iv) of industrial or domestic anhydrous compositions.

3. The process as claimed in claim 1, for treating a product that may be contaminated by bacteria of the Bcc complex, the product being selected from the group of i) of water or an aqueous composition, ii) of equipment for food, cosmetic or pharmaceutical purposes, iii) of solvents used in industrial or domestic preparations, and iv) of industrial or domestic anhydrous compositions.

4. The use process as claimed in claim 1, wherein formula (I) is such that:
R2 is chosen from hydrogen and methyl;
R3 represents (i) a C₁-C₁₀ alkyl radical; (ii) a C₂-C₁₀ alkenyl radical; or (iii) a hydroxyalkyl radical having the structure —CH₂—CH(OH)—R5 with R5 representing a linear C₁-C₁₀ alkyl radical.

5. The process as claimed in claim 1, wherein the compound of formula (I) is chosen from the following compounds:

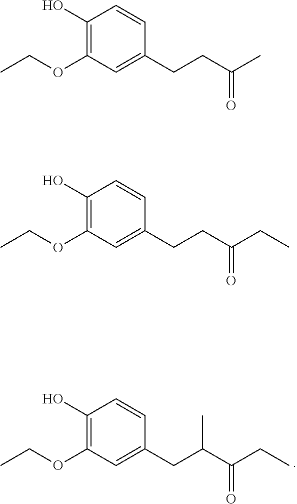

6. The process as claimed in claim 1, wherein the compound of formula (I) is 4-(3-ethoxy-4-hydroxyphenyl)butan-2-one having the following formula:

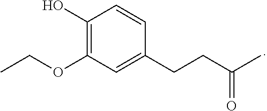

7. The process as claimed in claim 1, wherein the product is water or and aqueous composition and wherein the compound of formula (I), alone or as a mixture, is used in a proportion of at least 0.06% by weight relative to the total weight of water or of aqueous composition to be treated.

8. A process for treating a product selected from the group of water or an aqueous composition i) to prevent the contamination of said product by one or more microorganisms including bacteria of the Burkholderia cepacia complex, ii) or to treat said product that is contaminated by one or more microorganisms including bacteria of the Bcc complex, comprising at least one step of bringing a sample of the product to be treated or of a stream of water to be treated into contact with an amount effective for treating the product against bacteria of the Burkholderia cepacia complex of at least one compound of formula (I),
an organic or mineral acid or base salt thereof, a solvate thereof or a mixture thereof: 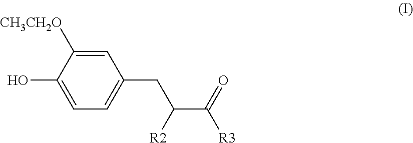
formula (I) wherein:
R2 represents a hydrogen atom or a methyl or ethyl radical;
R3 represents a linear C₁-C₁₂ alkyl radical (saturated), optionally substituted with a hydroxyl group; or a linear C₂-C₁₂ alkenyl radical (C═C unsaturated), optionally substituted with a hydroxyl group.

9. The process as claimed in claim 8, wherein the product, is chosen from domestic or industrial waters from industrial tanks, waters from aquatic media, swimming pool/spa waters, and air-conditioning system waters, and cosmetic products.

10. The process as claimed in claim 8, wherein the compound of formula (I), alone or as a mixture, is used in a proportion of at least 0.06% by weight, relative to the total weight of water or of aqueous composition to be treated.

11. A process for preserving a product that may be contaminated by bacteria of the Burkholderia cepacia complex, comprising a step of incorporating into said product, during the production thereof, or after the production thereof, and before storage, one or more compounds of formula (I),
an organic or mineral acid or base salt thereof, a solvate thereof or a mixture thereof: 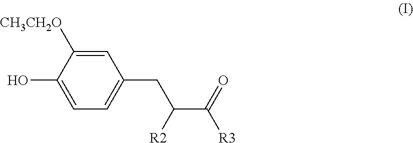
formula (I) wherein:
R2 represents a hydrogen atom or a methyl or ethyl radical;
R3 represents a linear C₁-C₁₂ alkyl radical (saturated), optionally substituted with a hydroxyl group; or a linear C₂-C₁₂ alkenyl radical (C═C unsaturated), optionally substituted with a hydroxyl group.

12. The process as claimed in claim 11, which comprises the step of incorporating one or more compounds of formula (I), during the phase of producing said product.

13. The process as claimed in claim 11, which comprises introducing into a preparation tank, beforehand, one or more compounds of formula (I) and then introducing, into said tank, the product to be treated and to be preserved, or the ingredients required to produce said product.

14. The process as claimed in claim 11, wherein each ingredient required for the production of the product has been treated beforehand with one or more compounds of formula (I), or each ingredient is introduced as a mixture with one or more compounds of formula (I); and then the product is preserved and made available to the a user in the presence of one or more compounds of formula (I).

15. The process as claimed in claim 11, wherein the process comprises the step of incorporating one or more compounds of formula (I) after the phase of producing said product, the product, once produced, is brought into contact with one or more compounds of formula (I), then the product is preserved and made available to a user in the presence of one or more compounds of formula (I).

16. An “anhydrous” composition comprising one or more compounds of formula (I) as defined in claim 1
an organic or mineral acid or base salt thereof, a solvate thereof or a mixture thereof: 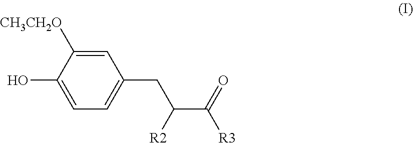
formula (I) wherein:
R2 represents a hydrogen atom or a methyl or ethyl radical;
R3 represents a linear C₁-C₁₂ alkyl radical (saturated), optionally substituted with a hydroxyl group; or a linear C₂-C₁₂ alkenyl radical (C═C unsaturated), optionally substituted with a hydroxyl group.

17. A composition as claimed in claim 16, comprises one or more ingredients chosen from i) anionic, nonionic, cationic or zwitterionic surfactants, ii) fatty substances, which are natural or synthetic, or derived from hydrocarbons, iii) anionic, nonionic, cationic or zwitterionic, associative or non-associative thickening or non-thickening polymers, iv) dyes, v) pigments, vi) fillers and vii) their mixture.

18. The process as claimed in claim 8, wherein formula (I) is such that:
R2 is chosen from hydrogen and methyl;
R3 represents (i) a C₁-C₁₀ alkyl radical; (ii) a C₂-C₁₀ alkenyl radical; or (iii) a hydroxyalkyl radical having the structure —CH₂—CH(OH)—R5 with R5 representing a linear C₁-C₁₀ alkyl radical.

19. The process as claimed in claim 8, wherein the compound of formula (I) is chosen from the following compounds:

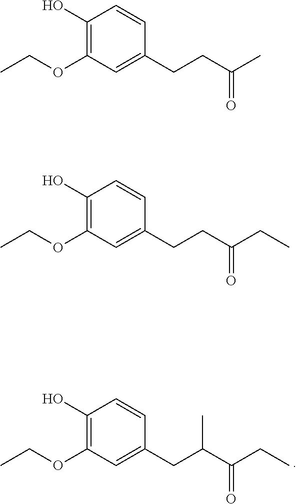

20. The process as claimed in claim 8, wherein the compound of formula (I) is 4-(3-ethoxy-4-hydroxyphenyl)butan-2-one having the following formula: [chemical structure]
